(19) United States
(12) Patent Application Publication
(10) Pub. No.: US 2023/0092457 A1
(43) Pub. Date: Mar. 23, 2023

(54) Device for Simulating Lower Jaw Activity

(71) Applicant: Tsung Chuan Liu, Taipei City (TW)

(72) Inventor: Tsung Chuan Liu, Taipei City (TW)

(21) Appl. No.: 17/481,291

(22) Filed: Sep. 21, 2021

(51) Int. Cl. A61C 11/08

(52) U.S. Cl. CPC A61C 11/088; A61C 11/084; A61C 2201/002

(57) ABSTRACT

The present invention is a device for simulating lower jaw activity. The device can be utilized in cooperation with an artificial intelligent lower jaw occlusion and attitude recording device. The device comprises a base, an upper jaw tooth mold, an opening and closing rotating platform, a left and right rotating platform, a horizontal rotating platform, a front-back shifting platform and a lower jaw tooth mold. The present invention makes it possible to make lower jaw moving forward and backward, opening and closing (pitch), tilting left and right (yaw), in place rotating (yaw), and etc. Then, according to the data obtained by the artificial intelligent lower jaw occlusion and attitude recording device, the lower jaw activities concluded through the reverse engineering by means of the present invention facilitates production of accurate intra-oral device, and can be applied to unknown complex actions in dental medical research, such as occlusion, bruxism, swallowing, sounding, and etc.

### BACKGROUND OF THE PRESENT INVENTION

### Field of Invention

The present invention relates to a technology of a device for simulating lower jaw activity, and more particularly, to a device for simulating lower jaw activity that is capable of performing movements including lower jaw forward and backward moving, opening and closing (pitch), tilting left and right (yaw), in place rotating (yaw), and etc., which is capable of enhancing the adaptation efficiency of the patient to the oral appliance as well as improving the occlusion efficiency.

### Description of Related Arts

Conventional mandible records are usually static and fixed-point recording through a wax sheet occlusion, which mode has no sensing, feedback, judgment, and coordination action, and does not represent a real functional action, but a static record made of a plaster model with an occlusion wax sheet. Nonetheless, biological function action should involve sensing, feedback, judgement, and coordination action, which in completely different from pure mechanical action.

Therefore, the prior art tends to produce a result with a large difference to the actual lower jaw motions, especially for the patients who lack tooth for many years, rendering mandibular bone variant, and mouth and face skewness, which greatly increase the difficulty in manufacturing the dental implant or the movable denture. After the manufacturing is completed, it still requires bite paper tests for several times, as well as a long-term adaptation period.

Therefore, an object of the present invention is to provide a device capable of simulating the lower jaw motions of moving forward and backward, opening and closing (pitch), tilting left and right (yaw), in place rotating (yaw), and etc., as well as coordinating with the data of the testing devices to reverse engineer for the lower jaw motions, so as to greatly improve the adaptation time of the patient to the intraoral device and enhance the occlusion efficiency.

### SUMMARY OF THE PRESENT INVENTION

An object of the present invention is to provide a device for simulating lower jaw activity, which mainly utilizes a simple structural design to allow the present invention to perform lower jaw motions like moving forward and backward, opening and closing (pitch), tilting left and right (yaw), in place rotating (yaw), and etc., and reverse engineer the lower jaw motions according to the data obtained through the test device of the patient, so as for producing more precise product of various intra-oral device and denture for the patient, which can therefore shorten the adaptation time of the intra-oral devices as well as enhance the occlusion efficiency of the patient. Besides, it may also be utilized in dentistry studies in uncertain complex actions, such as occlusion, bruxism, swallowing, sounding, and etc., to enhance the overall usability.

In order to achieve the above and other objects, the present invention provides a device for simulating lower jaw activity. The device can be utilized in cooperation with an artificial intelligent lower jaw occlusion and attitude recording device. The device comprises a base, an upper jaw tooth mold, an opening and closing rotating platform, a left and right rotating platform, a horizontal rotating platform, a front-back shifting platform and a lower jaw tooth mold. The base comprises a lifting screw perpendicularly arranged on a side thereof, a lifting motor arranged at an end of the lifting screw, and a roof arranged on the lifting screw close to the top end thereof. The upper jaw tooth mold is affixed below the roof of the base. The opening and closing rotating platform is arranged on top of the base. The left and right rotating platform is arranged on top of the opening and closing rotating platform. The horizontal rotating platform is arranged on top of the left and right rotating platform. The front-back shifting platform is arranged on top of the horizontal rotating platform. The lower jaw tooth mold is arranged on top of the front-back shifting platform. Accordingly, the lifting motor of the base can drive the opening and closing rotating platform, the left and right rotating platform, the horizontal rotating platform, and the front-back shifting platform to act to simulate movements of the upper jaw tooth mold and the lower jaw tooth mold.

According to a preferred embodiment, it further comprises a digital control system. The digital control system is capable of driving the lifting motor according to the test data. A holding rod is arranged on a side of the lifting screw. The opening and closing rotating platform is adapted to drive the upper jaw tooth mold and the lower jaw tooth mold to perform up and down rotations. The left and right rotating platform is adapted to drive the upper jaw tooth mold and the lower jaw tooth mold to perform in situ left and right rotations. The horizontal rotating platform is adapted to drive the upper jaw tooth mold and the lower jaw tooth mold to perform plane rotation. The front-back shifting platform is adapted to drive the upper jaw tooth mold and the lower jaw tooth mold to perform forward and backward movements. The test device is adapted for recording various types of data of the oral conditions of the patient.

According to a preferred embodiment, the artificial intelligent lower jaw occlusion and attitude recording device comprises: a head holder, at least three pairs of LEDs with different colors, and a microcomputer port mechanism. The head holder has a microcomputer arranged thereon. The three pairs of LEDs of different colors are positioned in the vicinity of the microcomputer at visible positions for the tester. A microcomputer port mechanism has a transmission unit, a pressure sensor, and an inertia measuring unit, wherein an end of the transmission unit is connected with the microcomputer, while the other end is connected with the pressure sensor and the inertia measuring unit, wherein the pressure sensor and the inertia measuring unit are placed in the tester's oral cavity. The transmission unit is a transmission wire or a wireless transmission port to electrically connect or wirelessly connect the microcomputer, the pressure sensor, and the inertia measuring unit.

### BRIEF DESCRIPTION OF THE DRAWINGS

FIG. 1 is a perspective view of a device for simulating lower jaw activity according to a preferred embodiment of the present invention.

FIG. 2 is a side view of the structure of a device for simulating lower jaw activity according to a preferred embodiment of the present invention.

FIG. 3 is a perspective view of a device for simulating lower jaw activity according to a preferred embodiment of the present invention.

FIG. 4 is a perspective view of an application scenario of a device for simulating lower jaw activity according to a preferred embodiment of the present invention.

### DETAILED DESCRIPTION OF THE PREFERRED EMBODIMENT

Other advantages and efficacy of the present invention will be readily apparent to those skilled in the art from the following specific and detailed description of embodiments of the present invention.

The embodiments of the present invention will be illustrated below with the figures, and it should be noted that the figures are merely illustrative of the principles of the present invention and are merely illustrative of the principles of the present invention and are not drawn to the exact quantities, shapes, and sizes of the elements. The forms, quantities, and rations of the elements in actual implementations of the present invention shall not be limited by the figures, but to meet the actual needs instead.

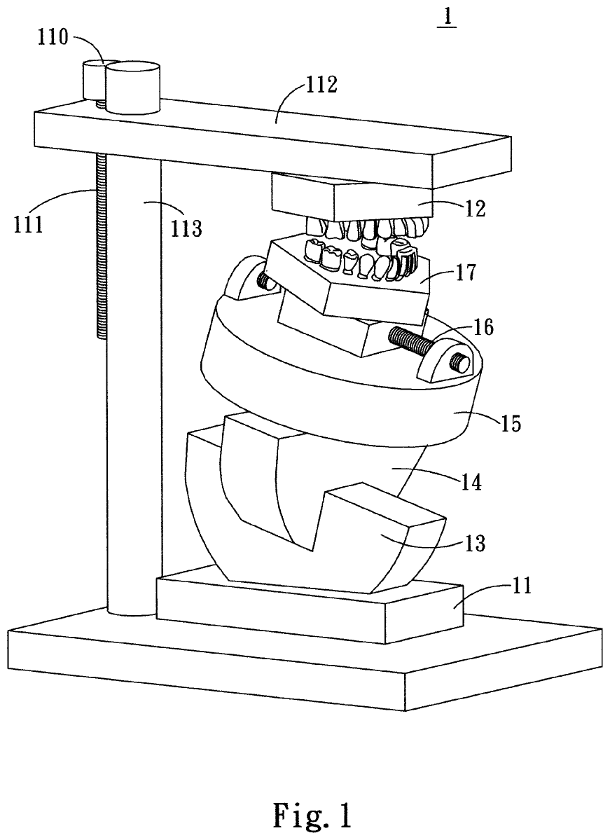

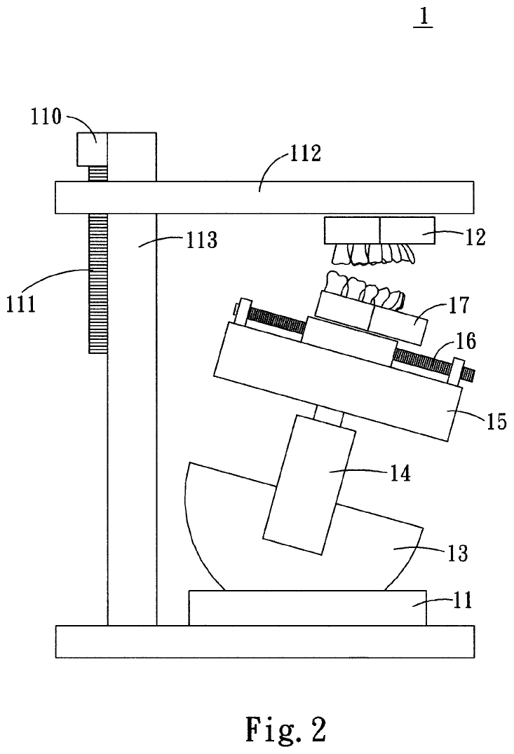

First, referring to FIG. 1-3, perspective views of an action, a side structure, and an exterior view of a device for simulating lower jaw activity according to a preferred embodiment of the present invention. As the figures illustrated, a device for simulating lower jaw activity 1 according to the present invention can be utilized in cooperation with an artificial intelligent lower jaw occlusion and attitude recording device. The device comprises a base 11, an upper jaw tooth mold 12, an opening and closing rotating platform 13, a left and right rotating platform 14, a horizontal rotating platform 15, a front-back shifting platform 16, and a lower jaw tooth mold 17. The base 11 comprises a lifting screw 111 perpendicularly arranged on a side thereof, a lifting motor 110 arranged at an end of the lifting screw 111, and a roof 112 arranged on the lifting screw 111 close to the top end thereof. The upper jaw tooth mold 12 is affixed below the roof 112 of the base 11. The opening and closing rotating platform 13 is arranged on top of the base 11. The left and right rotating platform 14 is arranged 14 on top of the opening and closing rotating platform 13. The horizontal rotating platform 15 is arranged on top of the left and right rotating platform. The front-back shifting platform 16 is arranged on top of the horizontal rotating platform 15. The lower jaw tooth mold 17 is arranged on top of the front-back shifting platform 16. Accordingly, the lifting motor 110 of the base 11 can drive the opening and closing rotating platform 13, the left and right rotating platform 14, the horizontal rotating platform 15, and the front-back shifting platform 16 to act to simulate movements of the upper jaw tooth mold 12 and the lower jaw tooth mold 17.

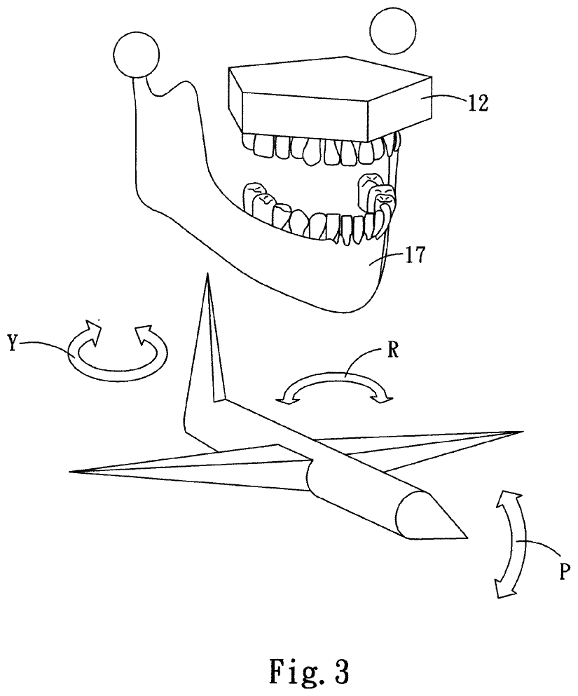

According to a preferred embodiment, it further comprises a digital control system. The digital control system is capable of driving the lifting motor 110 according to the test data. A holding rod 113 is arranged on a side of the lifting screw 111. The opening and closing rotating platform 13 is adapted to drive the upper jaw tooth mold 12 and the lower jaw tooth mold 17 to perform up and down rotations (P). The left and right rotating platform 14 is adapted to drive the upper jaw tooth mold 12 and the lower jaw tooth mold 17 to perform in situ left and right rotations (R). The horizontal rotating platform 15 is adapted to drive the upper jaw tooth mold 12 and the lower jaw tooth mold 17 to perform plane rotation (Y). The front-back shifting platform 16 is adapted to drive the upper jaw tooth mold 12 and the lower jaw tooth mold 17 to perform forward and backward movements (as illustrated in FIG. 3).

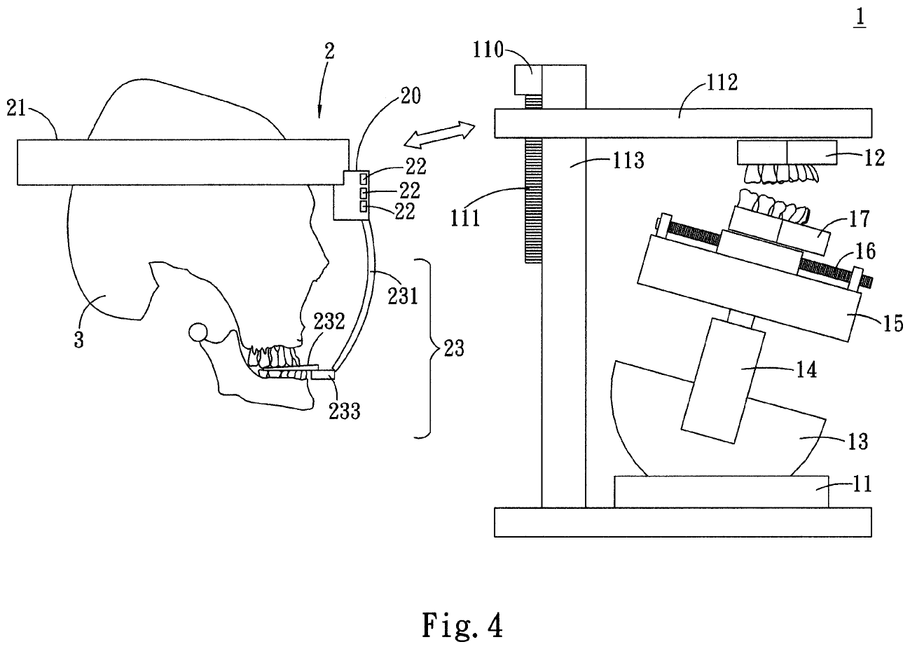

Referring to FIG. 4, a service mode of a device for simulating lower jaw activity according to a preferred embodiment of the present invention is illustrated. The device for simulating lower jaw activity 1 according to the present invention can be utilized in cooperation with an artificial intelligent lower jaw occlusion and attitude recording device 2. The artificial intelligent lower jaw occlusion and attitude recording device 2 comprises: a head holder 21, at least three pairs of LEDs 22, and a microcomputer port mechanism 23. The head holder 21 has a microcomputer 20 arranged on the front side thereof. The three pairs of LEDs 22 respectively have different colors and are positioned in the vicinity of the microcomputer 20 at visible positions for the tester. a microcomputer port mechanism 23, having a transmission unit 231, a pressure sensor 232, and an inertia measuring unit 233, wherein an end of the transmission unit 231 is connected with the microcomputer 20, while the other end is connected with the pressure sensor 232 and the inertia measuring unit 233, wherein the pressure sensor 232 and the inertia measuring unit 233 are placed in the tester 3's oral cavity.

Based on the above structure, in an actual use scenario, the head of a tester 3 is secured on a dentistry treatment chair or a specific place by means of a head holder 21. The microcomputer 20 on the head holder 21 is connected with the pressure sensor 232 and the inertia measuring unit 233 at the other side through the transmission unit 231. The device is affixed on the lower jaw dentition by means of wax sheet or other adhesive in the manner of not hindering occlusion. When the lower jaw acts based on the temporomandibular joints as the pivot points, it will link the inertia measuring unit 233. The inertia measuring unit 233 comprises a three-axis gyroscope with three-axis acceleration meter, which is capable of measuring the angular velocity and acceleration of the lower jaw in the three-dimensional space, and calculating the measured lower jaw posture change data in this way. When the upper and lower jaw teeth occluded, the pressure sensors 232 on the teeth on the left side and the right side of the lower jaw respectively generate occlusion pressure change data by means of the internal resistance changes. Then, all of the data will be analyzed and judged by the microcomputer 20, and the result will be fed back to the tester through light signals of the LEDs 22, which respectively display the strengths of the lower jaw occlusions of the two sides. Based on the settings, a green LED 22 shine for a light occlusion force, a yellow LED 22 shine for a medium occlusion force, and a red LED 22 shine for a hard occlusion force, such that the tester will be able to adjust for a proper occlusion force by him/herself based on the colors of the light signal. Therefore, it facilitates a biological cycle act of sensing, feedback, and coordination and is capable of fully recording the lower jaw postures and occlusion forces.

A dentist may utilize the data measured by the above artificial intelligent lower jaw occlusion and attitude recording device as well as rely on the device for simulating lower jaw activity 1 of the present invention to simulate lower jaw activities like moving forward and backward, opening and closing (pitch), tilting left and right (yaw), in place rotating (yaw), and etc., and reverse engineering the lower jaw motions through the present invention, so as for producing more precise products when producing any intra-oral device and denture. The product corresponds to personalized complex occlusion motions, so as to greatly shorten the adaptation period of the intraoral device for the patient, largely reduce the discomfort of the patient to the intraoral device, and enhance the occlusion efficiency.

Besides, the device for simulating lower jaw activity 1 according to the present invention may also be utilized in dentistry studies in uncertain complex actions, such as occlusion, bruxism, swallowing, sounding, and etc.

Compared with the prior art, the device for simulating lower jaw activity of the present invention mainly utilizes a simple structural design to perform lower jaw motions like moving forward and backward, opening and closing (pitch), tilting left and right (yaw), in place rotating (yaw), and etc., and to allow reverse engineer the lower jaw motions according to the data obtained through the external test devices, thereby achieving the purposes of making a more accurate denture or intraoral device product, greatly shortening the time for the patient to adapt to the denture and increasing the occlusion efficiency, which may be applied to various unknown complex actions in dental medical research to more satisfy the needs of the patients nowadays, so as to enhance the overall usability and convenience.

While the foregoing description and drawings have disclosed preferred embodiments of the present invention, it should be understood that various additions, modifications, and substitutions may be made to the preferred embodiments of the present invention without departing from the spirit and scope of the principles of the present invention as defined by the appended claims. One of ordinary skill in the art to which this disclosure pertains will appreciate that the present invention may be utilized with modifications in many forms, structures, arrangements, proportions, materials, elements, and components. Accordingly, the embodiments disclosed herein shall be considered as illustrative and not restrictive of the present invention. The scope of the present invention should be defined by the appended claims, and be intended to cover legal equivalents thereof, and not be limited to the previous description.

What is claimed is:

1. A device for simulating lower jaw activity, adapted for being utilized with an artificial intelligent lower jaw occlusion and attitude recording device, comprising:
a base, having a lifting screw perpendicularly arranged on a side thereof, a lifting motor arranged at an end of said lifting screw, and a roof arranged on said lifting screw close to the top end thereof;
an upper jaw tooth mold, affixed below the roof of said base;
an opening and closing rotating platform, arranged on top of said base;
a left and right rotating platform, arranged on top of said opening and closing rotating platform;
a horizontal rotating platform, arranged on top of said left and right rotating platform;
a front-back shifting platform, arranged on top of said horizontal rotating platform; and
a lower jaw tooth mold, arranged on top of said front-back shifting platform so as for said lifting motor of said base to drive said opening and closing rotating platform, said left and right rotating platform, said horizontal rotating platform, and said front-back shifting platform to move, to simulate movements of said upper jaw tooth mold and said lower jaw tooth mold.

2. The device for simulating lower jaw activity, as recited in claim 1, further comprising a digital control system, wherein said digital control system drives said lifting motor according to test data.

3. The device for simulating lower jaw activity, as recited in claim 1, wherein said lifting screw has a holding rod arranged on a side thereof.

4. The device for simulating lower jaw activity, as recited in claim 1, wherein said opening and closing rotating platform is configured to drive said upper jaw tooth mold and said lower jaw tooth mold to perform up and down rotations.

5. The device for simulating lower jaw activity, as recited in claim 1, wherein said left and right rotating platform is configured to drive said upper jaw tooth mold and said lower jaw tooth mold to perform in situ left and right rotations.

6. The device for simulating lower jaw activity, as recited in claim 1, wherein said horizontal rotating platform is configured to drive said upper jaw tooth mold and said lower jaw tooth mold to perform plane rotation.

7. The device for simulating lower jaw activity, as recited in claim 1, wherein said front-back shifting platform is configured to drive said upper jaw tooth mold and said lower jaw tooth mold to perform forward and backward movements.

8. The device for simulating lower jaw activity, as recited in claim 1, wherein the artificial intelligent lower jaw occlusion and attitude recording device is configured for recording various types of data of the oral conditions of the patient.

9. A device for simulating lower jaw activity, as recited in claim 1, wherein the artificial intelligent lower jaw occlusion and attitude recording device comprises:
a head holder, comprising a microcomputer arranged thereon;
at least of three pairs of LEDs with different colors, positioned in the vicinity of the microcomputer at visible positions for the tester;
a microcomputer port mechanism including a transmission unit, a pressure sensor, and an inertia measuring unit, wherein an end of the transmission unit is connected with the microcomputer, while the other end is connected with the pressure sensor and the inertia measuring unit, wherein the pressure sensor and the inertia measuring unit are adapted to be placed in an oral cavity of the tester.

10. The device for simulating lower jaw activity, as recited in claim 9, wherein the transmission unit is a transmission wire to electrically connect the microcomputer, the pressure sensor, and the inertia measuring unit.

11. The device for simulating lower jaw activity, as recited in claim 9, wherein the transmission unit is a wireless transmission port to wirelessly connect the microcomputer, the pressure sensor, and the inertia measuring unit.
